(19) United States
(12) Patent Application Publication
(10) Pub. No.: US 2013/0006292 A1
(43) Pub. Date: Jan. 3, 2013

(54) INTRA-GASTRIC DEVICE FOR ANIMALS

(75) Inventors: GUILLAUME NOEL, L'Arbresle (FR); ROMAIN LASSEUR, Bully (FR); ROLLAND ROUME, SOURCIEUX LES MINES (FR); JEAN-MICHEL VERD, SAINT-CHAMOND (FR); Pierre-André DENIS, Villeurbanne (FR)

(73) Assignee: ENDALIS, BRIGNAIS (FR)

(21) Appl. No.: 13/534,846

(22) Filed: Jun. 27, 2012

(30) Foreign Application Priority Data
Jun. 28, 2011 (FR) 1155731

(51) Int. Cl. A61M 29/02

(52) U.S. Cl. 606/192

(57) ABSTRACT

The invention relates to a device for the treatment of obesity or excess weight of a domestic or pet mammal, the device consisting of a balloon which is adapted to be placed in the animal's stomach and comprises a flexible envelope (E) sealed against air and alimentary and/or physiological liquids, as well as an inflating valve (V) hermetically sealed to the flexible envelope (E).

The invention also relates to the use of a flexible envelope (E) sealed against air and alimentary and/or physiological liquids and equipped with an inflating valve (V) to form a device for the treatment of obesity or excess weight of a domestic or pet mammal, this device being designed to be placed in this animal's stomach and filled in-situ with fluid.

This invention relates to the technical field of treating excess weight and even obesity in domestic animals or pets, particularly in domestic or pet mammals, such as dogs, cats, or horses.

In the field of treating dog obesity, it is known to implement diets based on dry dogfood nuggets specifically provided for this purpose. Such diets allow envisioning a weight loss on the order of 10% per month until the target weight is attained.

Even though these diets do effectively foster a weight loss, the time needed for the weight loss to be visible or sufficient can prove to be very long either due to the dog's general health or from the viewpoint of the dog owner who cannot really appreciate the diet's effectiveness. In addition, this weight loss may be too slow in some cases, particularly when the weight loss is necessary before the animal undergo surgery knowing that obesity is an obstacle to any surgical operation because of the vital risk that the animal may be exposed to.

Therefore, it became necessary to have a new treatment for obesity or excess weight of domestic animals or pets which allows obtaining a rapid loss of weight, while preserving the animal's health and well-being.

To attain this goal, the invention relates to a device for the treatment of obesity or excess weight of a domestic or pet mammal which consists of a balloon adaptable for placement in the animal's stomach and comprises a flexible envelope sealed against air and alimentary and/or physiological liquids, as well as an inflating valve hermetically sealed to the flexible envelope.

Such a device is known in the treatment of human obesity. However, its effectiveness with respect to animal excess weight or obesity problems was still not attained. In fact, in the case of a dog, for example, the stomach has the ability to distend up to six times its initial volume, while in humans the maximum distension is on the order of twice the initial volume. In addition, the instinctive behaviour of animals such as the dog consists of, a priori, eating as much as possible as long as food is available to stave off possible periods of food scarcity. Thus, it seemed established that, particularly in the case of a dog, the animal would not modify the quantity of the food it ingests even as a carrier of an intragastric balloon.

But the first tests conducted on dog surprisingly demonstrated that the animal with an intragastric balloon spontaneously reduces his daily absorbed intake, while preserving a normal behaviour and even acting as a player in the presence of stimulating objects. Thus, the spontaneous reduction of the daily absorbed intake allows a weight loss at a very clearly faster rate than with specific diets as will be subsequently shown.

Depending on the type of structure, the device for the treatment of obesity or excess weight of a domestic or pet mammal can also comprise an inflating tube whose movable distal end is placed on the inflating valve and has an adapted length so that the tube's proximal end extends inside the animal's mouth by passing through the oesophagus and the pharynx when the balloon is positioned in the animal's stomach.

According to a characteristic of the invention, the device for the treatment of obesity or excess weight of a domestic or pet mammal is adapted to be filled with gas or liquid or a mixture of liquid and gas. In a preferred aspect of implementation, the balloon is filled in a practically equal proportion with liquid and gas.

Still, according to another characteristic of the invention, the constituent flexible envelope of the device is made of two assembled discs of the same diameter D and adapted to be filled with fluid until practically forming a sphere with a diameter greater than or equal to 115% the diameter D of each disc.

The invention also relates to the use of such a device for the treatment of obesity or excess weight of a domestic or pet mammal and particularly for the treatment of obesity and excess weight of a dog, cat or horse.

The invention also relates to the use of a flexible envelope sealed against air and alimentary and/or physiological liquids and equipped with an inflating valve to form a device for the treatment of obesity or excess weight of a domestic or pet mammal, this device being designed to be placed in this animal's stomach and filled in-situ with fluid.

Within the framework of the invention, these uses can be made in conjunction with a diet based on:

either foods designed to control the excess weight of domestic animals or pets;

or foods designed to control the obesity of domestic animals or pets;

or foods designed to accompany the treatments of pathological disorders which can be generated by obesity of domestic animals or pets;

or mixtures of these foods.

Within the framework of these uses, the flexible envelope of the device can be filled with liquid and gas.

According to a structural alternative of the invention also liable to be implemented within the framework of the previous uses, the flexible envelope is made by assembling two discs with a diameter between 10 cm and 12 cm, and the device is designed to be placed in a large dog's stomach.

According to another structural alternative of the invention also liable to be implemented within the framework of the previous uses, the flexible envelope is made by assembling two discs with a diameter between 6 cm and 8 cm, and the device is designed to be placed in a medium dog's stomach.

According to still another structural alternative of the invention also liable to be implemented within the framework of the previous uses, the flexible envelope is made by assembling two discs with a diameter between 4 cm and 5.5 cm, and the device is designed to be placed in the stomach of a small dog or cat.

Still, according to another structural alternative of the invention also liable to be implemented within the framework of the previous uses, the flexible envelope is made by assembling two discs with a diameter between 12.5 cm and 14 cm, and the device is designed to be placed in the stomach of a horse or a very large dog.

Within the framework of the uses according to the invention, the fluid to fill the balloon can comprise a biocompatible colouring agent which is liable to colour urines in case of leaks.

The invention also relates to a process of treating obesity and excess weight of a domestic or pet mammal which consists of the following steps:

placement in the animal's stomach of a balloon including a flexible envelope sealed against air and alimentary and/or physiological liquids, as well as an inflating valve hermetically sealed to the flexible envelope;

filling of the balloon placed in the stomach with a fluid or a fluid mixture.

According to an implementation aspect of this process of treating obesity or excess weight of an animal, the balloon is placed in the mammal's stomach by passing through the natural tracts.

Within the framework of this aspect of implementation, the balloon is placed in the animal's stomach by passing through the animal's mouth, pharynx and oesophagus.

According to an implementation alternative of the process of treating obesity or excess weight of a domestic or pet mammal, the balloon is filled with liquid and gas.

Within the framework of this alternative, the balloon can be filled in a practically equal proportion with liquid and gas.

According to another alternative of the process of treating obesity or excess weight of a domestic or pet mammal in accordance with the invention, the liquid filling the balloon can comprise a biocompatible colouring agent which is liable to colour urines in case of leaks.

Still, according to another alternative of the process, in accordance with the invention of treating obesity or excess weight of a domestic or pet mammal, after the balloon is placed, a phase to feed the animal is implemented with:

either foods designed to control the excess weight of domestic animals or pets;

or foods designed to control the obesity of domestic animals or pets;

or foods designed to accompany the treatments of pathological disorders which can be generated by obesity of domestic animals or pets;

or mixtures of these foods.

Of course, the various characteristics, alternatives and aspects of realizing and using the invention, as well as the process in accordance with the invention, can be associated with one another according to various combinations, provided they are not incompatible or mutually exclusive.

In addition, various other characteristics of the invention emerge from the attached description referring to the drawings which show a non restrictive aspect of realizing a device for the treatment of excess weight or obesity of domestic animals or pets in accordance with the invention.

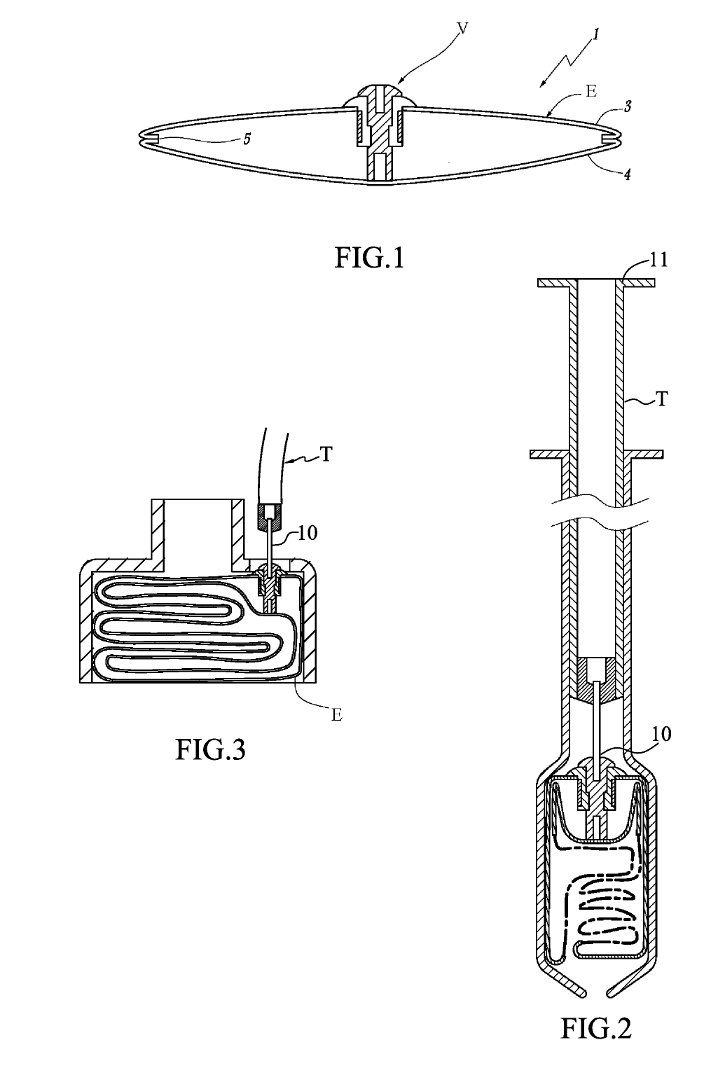

FIG. 1 is a schematic cross-section drawing of an intragastric balloon liable to be implemented within the framework of this invention.

FIG. 2 is an example of an intragastric balloon insertion structure according to the invention.

FIG. 3 is another example of an intragastric balloon insertion structure according to the invention including a plug designed to be adapted to the end of an endoscope.

An intragastric balloon liable to be used for the treatment of obesity of domestic or pet mammals, such as shown in FIG. 1 and globally designated by Reference 1, generally comprises a flexible envelope E, which is made of a material sealed against air and alimentary or physiological liquids, and is equipped with an inflating/aspirating valve V. According to the invention, any type of intragastric balloon can be implemented and particularly intragastric balloons certified for human uses.

Among the preferred candidate devices, the invention proposes to implement intragastric treatment devices, such as described by the demand WO2010/089468, which is incorporated here by reference and whose learning instruction perfectly describes the realization mode of an intragastric balloon and a placement device, so that it is not necessary to describe them more fully within the framework of this demand. In this light, it is possible to indicate, however, that according to the illustrated example, the envelope V is made by the assembling of two single-layer polyurethane discs 3 and 4 of a thickness e between 0.1 mm and 0.4 mm and, for example but not exclusively, on the order of 200 μm. The two discs are assembled end-to-end by means of a peripheral weld 5. The envelope E made by the assembling of the discs 3 and 4 is preferably narrowed through an opening 6 made in the upper disc 3 for the placement of the inflating valve V. The narrowing of the envelope 2 allows the peripheral weld 5 to be located inside the balloon 1, so that the balloon offers an external surface without bumps, thereby reducing the risks of irritation of the gastric wall after insertion.

The dimension of the balloon 1 and, therefore, in the present case, its constituent discs, is preferably chosen according to the animal's size in the stomach in which the intragastric balloon according to the invention must be inserted. Generally, the constituent material of the discs will be chosen so that the envelope made of two assembled discs of the same diameter D can be filled or inflated until practically forming a sphere with a diameter greater than or equal to 110% the diameter D of each disc.

For the treatment of cats or small dogs, that is, whose normal adult weight is less than or equal to 10 kilos, it may be chosen to implement the disks 3, 4 with a diameter between 4 cm and 5.5 cm. The intragastric balloon 1 thus made of these two discs can then be inflated until forming a sphere with a diameter between 5 cm and 6 cm.

For the treatment of medium dogs, that is, whose normal adult weight is greater than 10 kilos and less than or equal to 25 kilos, it may be chosen to implement the disks 3, 4 with a diameter between 6 cm and 8 cm. The intragastric balloon 1 thus made of these two discs can then be inflated until forming a sphere with a diameter between 10 cm and 11 cm.

For the treatment of large dogs, that is, whose normal adult weight is greater than 25 kilos and less than or equal to 45 kilos, it may be chosen to implement the disks 3, 4 with a diameter between 10 cm and 12 cm. The intragastric balloon 1 thus made of these two discs can then be inflated until forming a sphere with a diameter at least between 13 cm and 14 cm.

For the treatment of horses or very large dogs, that is, whose normal adult weight is greater than 45 kilos, it may be chosen to implement the disks 3, 4 with a diameter between 12.5 cm and 14 cm. The intragastric balloon 1 thus made of these two discs can then be inflated until forming a sphere with a diameter at least between 14 cm and 16 cm.

An intragastric balloon can be implemented in accordance with the invention as follows.

First of all, the animal may be administered a sedative or a general anesthesia. Prior to the placement of the balloon, a veterinary surgeon or an endoscopic operator preferably checks the general condition of the stomach by means of an endoscope. Then the balloon is placed by preferably passing by the upper natural tracts, namely, by the animal's mouth (dog or animal) and then successively by the pharynx and oesophagus until by the cardia to reach the stomach. The veterinary surgeon or the operator then proceeds to inflate the in-situ intragastric balloon in the stomach.

The balloon can be placed without any special accessories. The balloon can also be placed by means of an autonomous insertion structure, such as shown in FIG. 2, or else by means of an insertion structure including an endoscopic plug inside which the intragastric balloon is folded as shown in FIG. 3. To facilitate inflation, the intragastric balloon is associated with an inflating tube T on which a distal end 10 is mounted which is movable on the inflating valve. The tube T has an adapted length so that the proximal end 11 of tube T extends inside the animal's mouth by passing through the oesophagus and pharynx when the balloon is positioned inside the animal's stomach.

Treatment tests aimed at having two medium sized dogs no. 1 and no. 2, namely, two male Beagles three years old, lose a slight excess amount of weight, allowed obtaining the following results:

### Dog No. 1:

weight at start of test: 14.4 kg

weight 15 days after placement of the balloon: 13.3 kg

Throughout the entire test, the dog behaved normally as noted during the daily veterinarian clinical examinations and clearly tolerated the balloon very well. In 15 days, dog no. 1 lost 1.1 kg, that is, 7.6% of its initial weight.

### Dog No. 2:

weight at start of test: 13.8 kg

weight 10 days after placement of the balloon: 12,8 kg

Throughout the entire test, the dog behaved normally as noted during the daily veterinarian clinical examinations and clearly tolerated the balloon very well. In 10 days, dog no. 2 lost 1 kg, that is, 7.24% of its initial weight.
It seems that the implementation of a gastric balloon allows a much more rapid weight loss than that which would be obtained only by a diet.

To improve the performances of the treatment according to the invention, it is also proposed to associate the placement of the intragastric balloon with an adapted diet based on, for example:

either foods designed to control the excess weight of domestic animals or pets;

or foods designed to control the obesity of domestic animals or pets;

or foods designed to accompany the treatments of pathological disorders which can be generated by obesity of domestic animals or pets;

or mixtures of these foods.

Of course, various modifications can be made to the invention within the framework of the attached claims.

1. Device for the treatment of obesity or excess weight of a domestic or pet mammalian animal, comprising a balloon constructed and arranged for placement in the animal's stomach, the balloon including a flexible envelope sealed against air and alimentary and/or physiological liquids, and an inflating valve hermetically sealed to the flexible envelope.

2. Device according to claim 1, further comprising an inflating tube on which a distal end is mounted which is movable on the inflating valve and has an adapted length so that the proximal end of tube extends inside the animal's mouth by passing through the animal's esophagus and pharynx when the balloon is positioned inside the animal's stomach.

3. Device according to claim 1, wherein the balloon is constructed and arranged to be fillable with liquid and/or gas.

4. Device according to claim 1, wherein the flexible envelope is made of two assembled discs of the same diameter and adapted to be filled with fluid until practically forming a sphere with a diameter greater than or equal to 115% of the diameter of each disc.

5-13. (canceled)

14. A method of treating obesity or excess weight in a live domestic or pet mammalian animal, comprising:
passing a balloon having a construction and size suitable for placement in the animal's stomach through the upper alimentary tract of the animal and then into the animal's stomach; and
inflating the balloon within the animal's stomach with at least one inflating fluid.

15. The method according to claim 14, wherein the balloon comprises a flexible envelope sealed against air and alimentary and/or physiological liquids, and an inflating valve hermetically sealed to the envelope.

16. The method according to claim 15, wherein the envelope is constructed from a pair of disc-shaped elements having substantially the same diameter and sealingly joined at their respective peripheries.

17. The method according to claim 16, wherein the inflated envelope has a diameter at least about 115% the diameter of the disc-shaped elements.

18. The method according to claim 17, wherein the animal is a dog, cat, or horse, and the diameter of the disc-shaped elements is one of:
between 4 cm and 5.5 cm for use in a relatively small cat or dog;
between 6 cm and 8 cm for use in a relatively medium-sized dog;
between 10 cm and 12 cm for use in a relatively large dog; and
between 12.5 cm and 14 cm for use in a horse or relatively very large dog.

19. The method according to claim 17, wherein the disc-shaped elements are made from polyurethane.

20. The method according to claim 14, wherein the inflating fluid is one of a liquid, a gas, and a mixture of a liquid and a gas.

21. The method according to claim 20, wherein the inflating fluid is a mixture of a liquid and a gas in substantially equal parts.

22. The method according to claim 20, wherein the inflating fluid further includes a biocompatible coloring agent for coloring the animal's urine in a visually perceptible manner if the inflating fluid leaks from the balloon into the animal's digestive tract, thereby signaling a leak of inflating fluid.

23. The method according to claim 14, further comprising providing the animal with a controlled diet of one or more of:
food for controlling obesity or excess weight in a domestic or pet animal; and
food for supporting treatment of pathological animal disorders associated with excess weight or obesity.
